(19) United States
(12) Patent Application Publication
(10) Pub. No.: US 2024/0261410 A1
(43) Pub. Date: Aug. 8, 2024

(54) SODIUM DEOXYCHOLATE BILE ACID AS ANTIBIOTIC CARRIER

(71) Applicant: The United States of America, as represented by the Secretary of Agriculture, Washington, DC (US)

(72) Inventors: NEIL P. PRICE, EDELSTEIN, IL (US); MICHAEL A. JACKSON, MORTON, IL (US)

(21) Appl. No.: 18/166,114

(22) Filed: Feb. 8, 2023

(51) Int. Cl. A61K 47/28; A61K 9/107; A61K 31/7072; A61P 31/04

(52) U.S. Cl. CPC A61K 47/28; A61K 9/107; A61K 31/7072; A61P 31/04

(57) ABSTRACT

The disclosure provides stable aqueous formulations comprising an antibiotic and sodium deoxycholate (DOC), and optionally further comprising at least one carrier and/or excipient. The disclosure also provides methods for preparing such aqueous formulations, and methods for using such aqueous formulations to treat, attenuate, or prevent bacterial infection in a subject.

### FIELD OF THE INVENTION

The disclosure relates to aqueous antibiotic formulations comprising sodium deoxycholate (DOC), and optionally at least one carrier and/or excipient.

### BACKGROUND OF THE INVENTION

Over the past three decades, clinical interest in new antibiotics has increased due to the emergence of drug-resistant bacterial pathogens, and this is now a major priority of the World Health Organization (WHO) and of the U. S. Department of Agriculture (USDA).

The tunicamycins (TUN) are a group of N-acylated nucleotide antibiotics produced in fermentation by several Streptomyces strains under the influence of the tun biosynthetic genes. The natural TUN compounds contain an unusual 11-carbon dialdose sugar called tunicamine which at the 1-position is N-linked to uracil, and at the 11-position is O-linked to an N-acetylglucosamine residue. This tunicaminyl-uracil core structure is N-acylated with various branched chain fatty acids with differing carbon chain lengths (C14-C18) and terminal branching patterns (iso-, anteiso-, or straight chain) resulting in a variety of tunicamycin N-acyl variants as seen in FIG. 1. The native tunicamycins are potent inhibitors of the polyprenyl-phosphate N-acetylhexosamine-1-phosphate-transferase (PNPT) enzymes superfamily, which are essential in both eukaryotes and bacteria. In eukaryotes, the PNPT homolog DPAGT1 (human GPT homolog) catalyzes the first step in protein N-glycosylation and inhibition by tunicamycins which results in misfolding of glycoproteins during their biosynthesis, and subsequently a lethal unfolded protein shock cellular response. Native tunicamycins are toxic to eukaryotic cells via this mechanism, but are a commonly used biochemical tool to study N-glycoprotein biosynthesis. The bacterial response to tunicamycins is also mediated via PNPT-binding, but bacterial PNPT family members (MraY, WbcO, WecA) catalyzes the formation of peptidoglycan and teichoic acid cell wall components, a crucial step for bacterial growth and cell division.

Thus, tunicamycin showed severe toxicity in eukaryotic cells, and is used to study the endoplasmic reticulum stress response as a bioprobe. Recent studies have compared the structures of tunicamycin bound to DPAGT1 and MraY enabling the design of analogues with altered lipid side chains imparting selectivity for MraY over DPAGT1. Chemically modifying tunicamycin, hydrogenating the N-acetyl double bond, or hydrogenating both the N-acetyl and uridyl double bonds have also been reported to impart less toxic to eukaryotic cells, while retaining their antibacterial activity. U.S. Pat. No. 10,513,533 reports a group of modified tunicamycins referred to as TunR1, TunR2, and TunR3. The chemical structure of single reduced tunicamycins (referred to as “Tun-R1”) is depicted in FIG. 2A. The chemical structure of double reduced tunicamycins (referred to as “Tun-R2”) is depicted in FIG. 2B. These molecules have selective biological activity against bacteria, and considerably reduced activity against eukaryotes. The TunR2 compounds, in which the tunicaminyl N-uracil group is replaced by N-linked 5,6-dideoxyuracil, are essentially non-toxic to eukaryotes whilst maintaining potent antibacterial properties, and including the enhancement ability in combination with several, well-established beta-lactam antibiotics. Omega-alicyclic tunicamycins with reduced activity against eukaryotes while maintaining activity against procaryotes have also been prepared. The chemical structures of omega-alicyclic tunicamycins, branched chain acyl groups, and omega-alicyclic acyl groups used in their preparation are shown in FIG. 3A to FIG. 3C.

However, the clinical adoption of modified tunicamycins has been limited to date by their poor water solubility or the availability of an aqueous-based carrier, suitable for intravenous administration in large animals. Existing solubilizing agents such as methanol and dimethyl sulfoxide (DMSO) present with high toxicity, which has hindered toxicology and pharmacological studies in animals.

Thus, compositions comprising an aqueous-based carrier and an antibiotic with inferior water solubility that is safe in animals is urgently needed.

### SUMMARY OF THE INVENTION

Provided herein are aqueous formulation comprising an antibiotic and sodium deoxycholate (DOC), and optionally at least one carrier and/or excipient, methods for preparing such aqueous formulations, and methods for using such aqueous formulations to treat a subject in need thereof.

In an embodiment, the invention relates to an aqueous formulation comprising at least one antibiotic and sodium deoxycholate (DOC), and optionally comprising at least one carrier and/or excipient. In some embodiments of the invention, the at least one antibiotic has poor water solubility, and is a tunicamycin, a modified tunicamycin, capazamycin, liposidomycin, muraminomicin, polymyxin B, nikkomycin Z, or a lipophilic antifungal polyene antibiotic, such as amphotericin B, candicidin, filipin, hamycin, natamycin, nystatin, rimocidin, or a mixture thereof. In some embodiments of the invention, the at least one antibiotic is a modified tunicamycin, capazamycin, liposidomycin, muraminomicin, or a mixture thereof. In some embodiments of the invention the modified tunicamycin is TunR2.

In an embodiment, the invention relates to a method for treating a subject in need thereof, the method comprising administering to the subject an effective amount of an aqueous formulation comprising at least one antibiotic and DOC, and at least one carrier and/or excipient.

In an embodiment, the invention relates to a stable antibiotic/DOC complex. In some embodiments of the invention the antibiotic in the antibiotic/DOC complex is a tunicamycin, a modified tunicamycin, capazamycin, liposidomycin, muraminomicin, polymyxin B, nikkomycin Z, or a lipophilic antifungal polyene antibiotic. The lipophilic antifungal polyene antibiotic may be amphotericin B, candicidin, filipin, hamycin, natamycin, nystatin, rimocidin, or a mixture thereof. In some embodiments of the invention, the at least one antibiotic in the antibiotic/DOC complex is a modified tunicamycin, capazamycin, liposidomycin, muraminomicin, or a mixture thereof.

### BRIEF DESCRIPTION OF THE DRAWINGS

FIG. 1 depicts structures of tunicamycin branched variants as described in NPJ Price et al. (2021, “Branched Chain Lipid Metabolism As a Determinant of the N-Acyl Variation of Streptomyces Natural Products,” ACS Chem. Biol. 16:116-124).

FIG. 2A to FIG. 2C depict chemical structures of reduced tunicamycins. FIG. 2A shows the chemical structure of single reduced tunicamycins (referred to as “TunR1”) in which the fatty acid acyl chain's length can vary and its 2′″,3′″-double bond is reduced. FIG. 2B shows the chemical structure of double reduced tunicamycins (referred to as “TunR2”) in which the fatty acid acyl chain's length can vary and its 2′″,3′″-double bond and the uracil 5,6-double bond are both reduced. FIG. 2C shows the chemical structure of a naturally-occurring streptovirudin (“TunR3”) in which the uracil 5,6-bond is fully reduced, and the fatty acid acyl chain contains a double bond at the 2′″,3′″ position.

FIG. 3A to 3C depict chemical structures of omega-alicyclic tunicamycins, branched chain acyl groups, and omega-alicyclic acyl groups used in the preparation of omega-alicyclic tunicamycins. FIG. 3A shows tunicamycin-omega-X, TunR1-omega-X, and TunR2-omega-X.

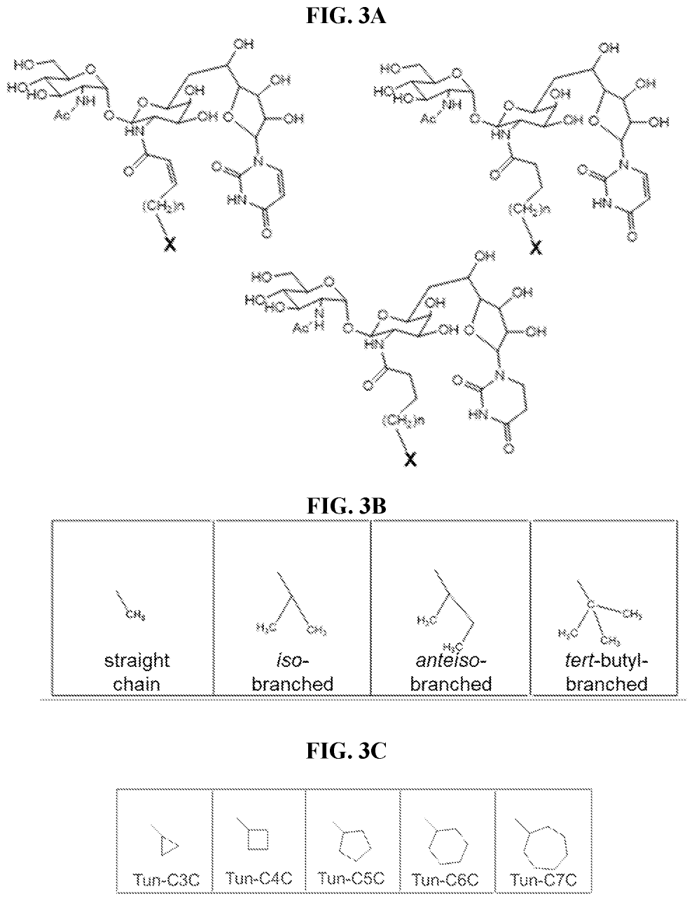

FIG. 3B shows the branched chain acyl groups that may be inserted at the “X” position of omega-tunicamycins. FIG. 3C alicyclic acyl groups that may inserted at the “X” position of omega-tunicamycins.

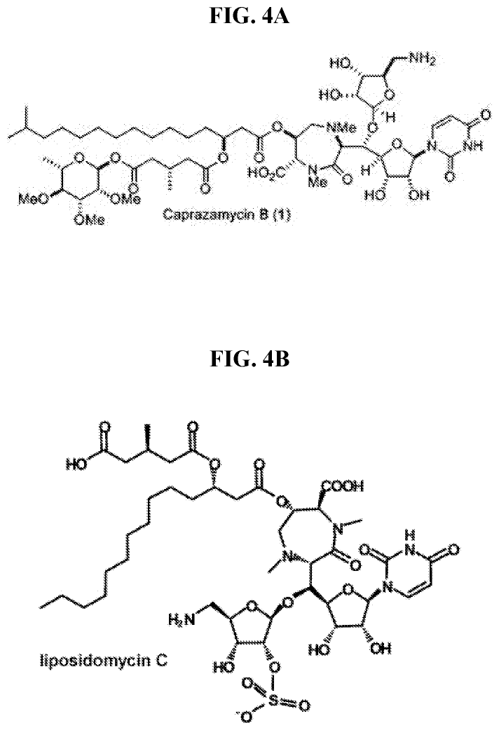

FIG. 4A to FIG. 4C depict chemical structures of capazamycin, liposidomycin, and muraminomicin. FIG. 4A shows the chemical structure of caprazamycin. FIG. 4B shows the chemical structure of liposidomycin. FIG. 4C shows muraminomicin.

FIG. 5A and FIG. 5B depict the chemical structures of sodium deoxycholate and TunR2. FIG. 5A shows the chemical structure of sodium deoxycholate. FIG. 5B shows the chemical structure of TunR2.

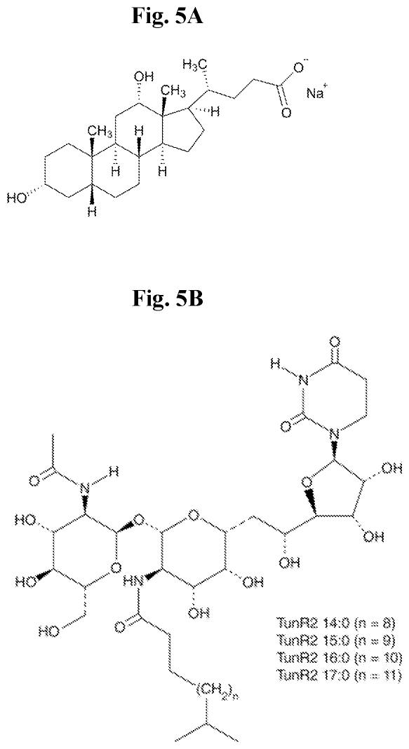

FIG. 6A to FIG. 6D depict images of the solubilization stages of native tunicamycin and modified TunR1 and TunR2 in water with sodium deoxycholate. FIG. 6A shows pictures of the antibiotics after the addition of water and sodium deoxycholate. FIG. 6B shows pictures of the antibiotics after heating for 5 minutes at 50° ° C. FIG. 6C shows pictures of the antibiotics after heating for 10 minutes at 50° C. FIG. 6D shows pictures of the antibiotics after cooling at room temperature for 18 hours. 1: native tunicamycin; 2: TunR1; 3: TunR2.

FIG. 7A to FIG. 7C depict images of the complexation gelling of TunR2-DOC micellar structure. FIG. 7A shows a picture of an equimolar solution of TunR2 and DOC (10 mM) in 1 mL deionized water. FIG. 7B shows a picture of gelling after 24 hour freeze-thawing with the tube held vertically. FIG. 7C shows a picture of gelling after 24 hour freeze-thawing with the tube held horizontally.

FIG. 8A and FIG. 8B depict graphs of MALDI-TOF/MS spectra of TunR2 dissolved in aqueous solution with sodium deoxycholate. FIG. 8A shows an MS spectrum acquired Sep. 30, 2022. FIG. 8B shows an MS spectrum acquired Nov. 3, 2022, after the sample had been left at room temperature for 34 days.

FIG. 9A and FIG. 9B depict graphs of the Diffusion-Orientated Spectra (DOSY-NMR) of the TunR2-DOC complex and sodium deoxycholate. FIG. 9A shows the DOSY-NMR spectra of the TunR2-DOC complex (diffusion constant (log D)=−10.28 m/s2). FIG. 9B shows the DOSY-NMR spectra of sodium deoxycholate (log D=−9.66 m/s2). The log D of residual deuterated water (HDO) and methanol (MeOD) are also indicated.

FIG. 10 depicts an image of a microtiter plate used to analyze the minimal inhibitory ability and the minimal biofilm inhibitory concentration of the TunR2-DOC against the model TB organism Mycobacterium smegmatis. The minimal inhibitory concentration (MIC, 1.25 ug/mL) is as denoted by solid lines and a solid arrow, and the minimal biofilm inhibitory concentration (MBIC, 0.625 ug/mL) is denoted by dashed lines and a dashed arrow. Control lanes are TYG medium only, and TYG+DOC gradient (2.5-0.02 ug/mL).

FIG. 11 depicts an image of a microtiter plate used to analyze the minimal inhibitory ability of TunR2-DOC in water and TunR2 in DMSO against Mycobacterium avium subsp. paratuberculosis, the mycobacterial pathogen of Johne's disease in cattle. The arrow above the microtiter plate indicates the measured MIC, 4 μg/mL.

### DETAILED DESCRIPTION

Disclosed herein are aqueous antibiotic formulations comprising sodium deoxycholate bile acid, that can comprise at least one pharmaceutically-acceptable carrier, and/or excipient.

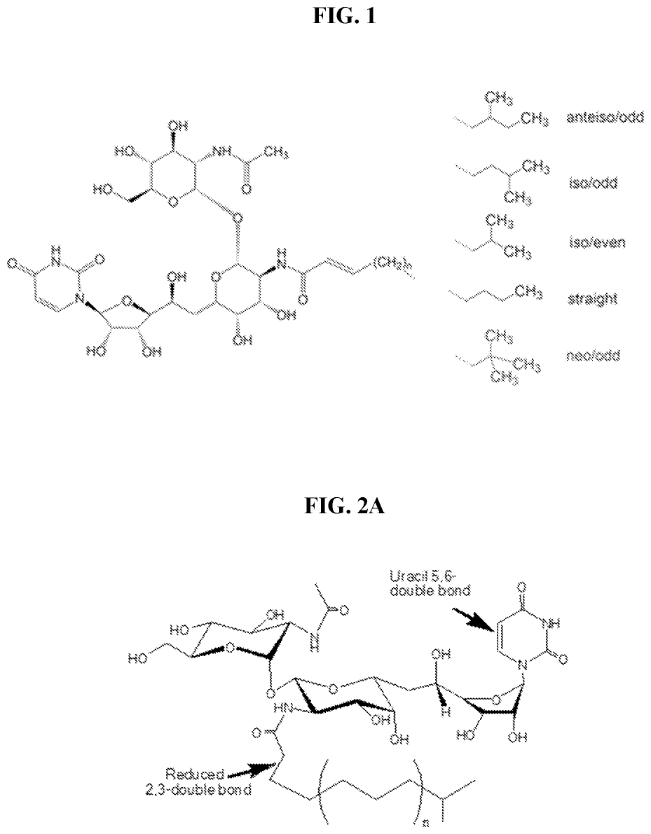

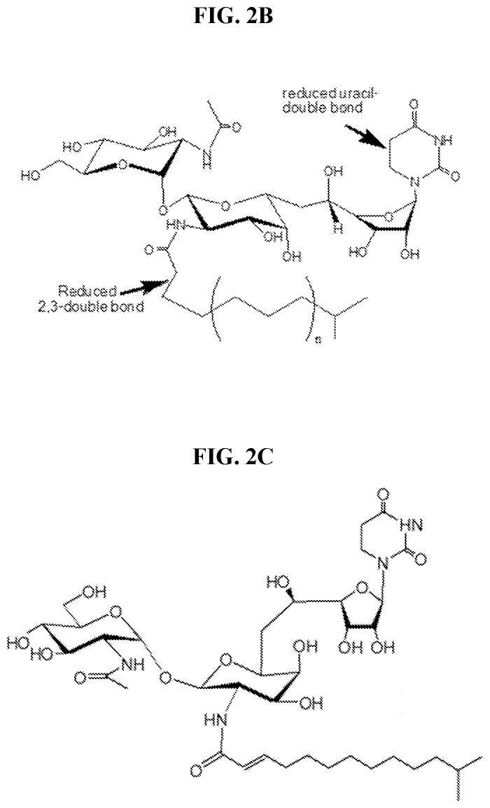

Over the past three decades, clinical interest in new antibiotics has increased due to the emergence of drug-resistant bacterial pathogens, and this is now a major priority of the World Health Organization (WHO) and of the U. S. Department of Agriculture (USDA). FIG. 1 depicts structures of tunicamycin branched variants as described in NPJ Price et al. (2021, “Branched Chain Lipid Metabolism As a Determinant of the N-Acyl Variation of Streptomyces Natural Products,” ACS Chem. Biol. 16:116-124). TunR2 (the structure of which is depicted in FIG. 2B) is a modified tunicamycin developed by the USDA as a novel mode-of-action antibiotic and is currently in animal trials for defense against Gram-positive pathogens and mycobacterial infections such as Johne's disease in cattle. The mechanism of action of TunR2 involves the disruption of peptidoglycan and teichoic acid assembly, both major component of the cell wall of Gram-positive bacteria, and also a well characterized target for the beta-lactam class of antibiotics. Indeed, as well as having antibacterial activity, TunR2 is also a potent enhancer of the beta-lactam family, including penicillins, cephalosporins, penems, and cephems.

The tunicamycins are a group of N-acylated nucleotide antibiotics produced in fermentation by several Streptomyces strains under the influence of the tun biosynthetic genes. The natural TUN compounds contain an unusual 11-carbon dialdose sugar called tunicamine which at the 1-position is N-linked to uracil, and at the 11-position is O-linked to a N-acetylglucosamine residue (FIG. 1). This tunicaminyl-uracil core structure is N-acylated with various branched chain fatty acids with differing carbon chain lengths (C14-C18) and terminal branching patterns (iso-, anteiso-, or straight chain) resulting in a variety of tunicamycin N-acyl variants. The native tunicamycins are potent inhibitors of the polyprenyl-phosphate N-acetylhexosamine-1-phosphate-transferase (PNPT) enzymes superfamily, which are essential in both eukaryotes and bacteria. In eukaryotes, the PNPT homolog DPAGT1 (human GPT homolog) catalyzes the first step in protein N-glycosylation and inhibition by tunicamycins results in misfolding of glycoproteins during their biosynthesis, and subsequently a lethal unfolded protein shock cellular response. Native tunicamycins are toxic to eukaryotic cells via this mechanism, but are a commonly used biochemical tool to study N-glycoprotein biosynthesis. The bacterial response to tunicamycins is also mediated via PNPT-binding, but bacterial PNPT family members (MraY, WbcO, WecA) catalyzes the formation of peptidoglycan and teichoic acid cell wall components, a crucial step for bacterial growth and cell division. The inventors have recently reported a group of modified tunicamycins called TunR1 and TunR2 (FIG. 2A and FIG. 2B) that have selective biological activity against bacteria, and considerably reduced activity against eukaryotes. The TunR2 compounds, in which the tunicaminyl N-uracil group is replaced by N-linked 5,6-dideoxyuracil, are essentially non-toxic to eukaryotes whilst maintaining potent antibacterial properties, and including the enhancement ability in combination with several, well-established beta-lactam antibiotics.

TunR2, and to a lesser extent, also TunR1, have considerable promise as novel mode-of action antibiotics, especially against pathogenic Gram positive and mycobacterial strains. Of particular importance to veterinary medicine are new control agents for Mycobacterium avium subsp. paratuberculosis (MAP) strains, the causative agents of Johne's disease in livestock. Johne's is a wasting disease in sheep, goats, and cattle, and the causative agent is closely related to the TB pathogen in humans. However, to date the clinical adoption of TunR2 or other modified tunicamycins has been limited by the lack of availability of an aqueous-based carrier, suitable for intravenous administration in large animals. Poor water solubility of the tunicamycins, coupled with the high toxicity of existing solubilizing agents such as methanol or dimethyl sulfoxide (DMSO) has prevented toxicology and pharmacological studies in animals.

Moreover, sodium deoxycholate sulfate (SDCS)-based formulations for inhalation could improve stability, solubility, bioactivity, and safety by reducing toxicity to macrophage and embryonic kidney cells. Our previous work on SDCS revealed that PMB loaded SDCS neutralized and detoxified the lipopolysaccharides (LPS) micelles with minimal toxicity, in vitro (S. Madhumanchi, et al., 2019, “Binding interactions of bacterial lipopolysaccharides to polymyxin B in an amphi-philic carrier ‘sodium deoxycholate sulfate,” Colloids Surf. B Biointerfaces 182: 110374). So, it is quite evident that SDCS is a safe carrier for drug development. Therefore, SDCS was the carrier used here with polymyxin B (PMB) as our molecule of interest to develop a new formulation of PMB. In this study, the inventors developed and characterized a new formulation of TunR2 carried with sodium deoxycholate to investigate the physicochemical properties, the stability in aqueous solutions, and in vitro efficiency and cytotoxicity of TunR2-DOC micelles.

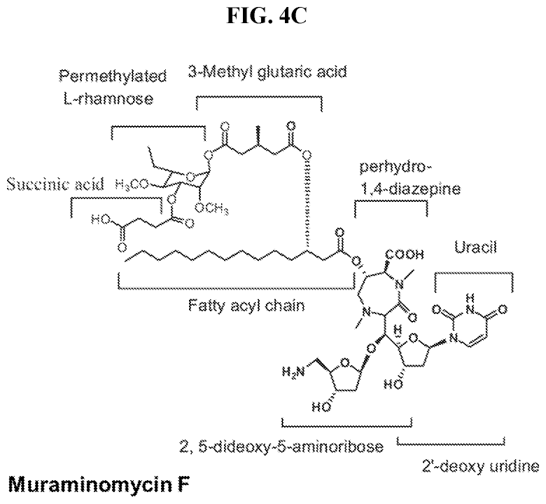

The inventors surprisingly found that addition of deoxycholate to modified tunicamycins form stable gels that may be dissolved in water. Prior to the instant disclosure, it was not known if addition of sodium deoxycholate to a modified tunicamycin would allow the formation of an aqueous stable formulation. In the instant disclosure the formulation of TunR2 with DOC has been prepared and analyzed. It is possible to use the same methodology to produce aqueous solutions of any antibiotic that presents poor water solubility. Thus, it is envisioned that the methods taught herein may be applied to other molecules such as capazamycin, liposidomycin, and muraminomicin. The chemical structure of caprazamycin is shown in FIG. 4A, the chemical structure of liposidomycin is shown in FIG. 4B, and the chemical structure of muraminomicin is shown in FIG. 4C.

Cholate is a common bile salt, produced in the bile duct, and which functions in the body to complex and solubilize biological lipids, such as cholesterol and phospholipids. Deoxycholic acid (DOC) is one of the secondary cholic acids, which are metabolic byproducts of intestinal bacteria. It has a steroidal-type structure, unlike the typical amphiphilic surfactants, with a concave, hydrophilic face (the α-plane) and a hydrophobic, convex face (β-plane) as determined by the orientation of hydroxy and methyl groups on the molecular surface. At low concentration, DOC forms head-to-tail dimers, with the two lipophilic β-planes orientated together and with the α-planes exposed to the aqueous medium. This β-face-to-β-face arrangement provides a cavity that allows lipophilic molecules to bind and hence to be solubilized in an aqueous environment. The NMR T₂ relaxation rates measured for a dilute DOC/cetyltrimethylammonium bromide (CTAB) solution show that this forms 1:1 mixed micelles as anti-parallel, back-to-back aggregates held together through hydrophobic interactions. The pairs begin to form above concentrations of 2.4 mM which is in good agreement with the stable DOC-TunR2 complex observed by DOSY-NMR at 10 mM concentrations. Not wishing to be bound by any particular theory, the model of this aggregation behavior is therefore similar to that proposed for the binding and transport of DOC-cholesterol micelles in the blood stream, and therefore provided desirable properties for the oral or intravenous administration of tunicamycin-based drugs.

The inventors surprisingly found that TunR2-DOC micelles are formed when TunR2 is mixed with DOC. These micellular formulations were found to be stable after reconstitution in aqueous solution, and did not change the antibacterial activity of the TunR2 antibiotic. This preparation is suitable for water-soluble tunicamycin formations, avoiding the need for health hazard solvents such as DMSO. Thus, these micelles are useful for drug formulation, particularly for the intravenous administration of TunR2 antimicrobials in veterinary medicine.

Sodium deoxycholate (deoxycholic acid) is a water soluble, bile acid formed by bacterial action from cholate. Deoxycholic acid acts as a detergent to solubilize fats for intestinal absorption and is reabsorbed itself. Deoxycholic acid is commonly used in protein methods. Most frequently it is used as a component of cell lysis buffers, but has also been used for liposome preparation, isolation of membrane proteins and lipids, to prevent non-specific binding in affinity chromatography, and as a cell culture media supplement.

Besides the tunicamycin-type antibiotics that are described herein, use of deoxycholate may be applicable to other antibiotics that have poor water solubility. These may include capazamycin, liposidomycin, muraminomicin, polymyxin B, nikkomycin Z, or a lipophilic antifungal polyene antibiotic. The lipophilic antifungal polyene antibiotic may be amphotericin B, candicidin, filipin, hamycin, natamycin, nystatin, and rimocidin. Other antibiotics found with low water solubility are also anticipated to be solubilized in water or water-based buffers by the application of DOC.

U.S. Pat. No. 11,458,145 discloses the preparation of a 30 mg/mL meloxicam nanocrystal aqueous dispersion consisting of 30 mg meloxicam nanocrystals; povidone; sodium deoxycholate; an excipient; and water.

In an embodiment, the invention relates to an antibiotic formulation comprising sodium deoxycholate (DOC), and optionally at least one carrier and/or excipient. In some embodiments of the invention, the at least one carrier and/or excipient in the antibiotic formulation comprising DOC is sodium deoxycholate sulfate (SDCS) or polymyxin B (PMB).

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

As used herein, the term “about” is defined as plus or minus ten percent of a recited value. For example, about 1.0 g means 0.9 g to 1.1 g.

The terms “optional” and “optionally” are used interchangeably herein and mean that the subsequently described substance, event, or circumstance may or may not occur, and that the description includes instances in which the described substance, event, or circumstance occurs and instances where it does not. For example, the phrase “optionally at least one excipient” means that the aqueous formulation comprising an antibiotic and DOC may or may not contain at least one excipient, and that the Examples include formulations that contain and do not contain an added excipient.

As used herein, an immunological response refers to a development in the host subject of a cellular and/or antibody-mediated immune response to a composition of interest.

The term “effective amount” of a compound, formulation, or property as provided herein is meant such amount as is capable of performing the function of the compound, formulation, or property for which an effective amount is expressed. As is pointed out herein, the exact amount required will vary from process to process, depending on recognized variables such as the compound, formulation, or properties employed, and the various internal and external conditions observed as would be interpreted by one of ordinary skill in the art. Thus, it is not possible to specify an exact “effective amount,” though preferred ranges have been provided herein. An appropriate effective amount may be determined, however, by one of ordinary skill in the art using only routine experimentation.

The term “carrier” as used herein includes carrier materials such as those described below. The carrier can be any physiologically or pharmaceutically acceptable carrier. The carrier may be a liquid or a solid. As is known in the art, the carrier to be used refers to a substrate such as a mineral oil, a paraffin, a silicon oil, water, a membrane, a sachet, a disk, a rope, a vial, a tube, a septa, a resin, a hollow fiber, a microcapsule, a filter, a gel, a fiber, a natural polymer, a synthetic polymer, an elastomer, or the like. All of these substrates may be used for an aqueous formulation comprising an antibiotic and DOC, optionally SDCS, and optionally at least one excipient. Suitable carriers are well-known in the art and are selected in accordance with the ultimate application of interest.

Pharmaceutically acceptable carriers include an aqueous solution, a glycol, an alcohol, a ketone, an ester, a hydrocarbon, a halogenated hydrocarbon, a polyvinyl chloride, and mixtures thereof. In addition, the carrier may be a solid such as a clay, a laminate, a cellulosic matrix, a rubber matrix, a synthetic polymer matrix, or the like.

As used herein, the term “excipient” refers to an inactive substance that serves as the vehicle or medium for a drug or other active substance. Examples of excipients are coloring agents, preservatives, fillers, and the like.

Any effective route of administration may be utilized to deliver the aqueous formulations of the invention, such as, for example, orally, nasally, enterally, parenterally, intramuscularly or intravenously, subcutaneously, intradermally, rectally, vaginally, topically, ocularly, pulmonarily, or by contact application. From a practical standpoint, oral, (intra)nasal, parenteral (IM, SubQ, and perhaps intradermal) and ocular may be preferred. In some embodiments, aqueous formulations of the invention may be injected (e.g., via intramuscular, intraperitoneal, intradermal and/or subcutaneous routes); or delivered via the mucosa (e.g., to the oral/alimentary, respiratory, and/or genitourinary tracts). Intranasal administration of such formulations may be particularly useful in some contexts. In some embodiments of the invention, it may be desirable to administer different doses of an aqueous formulation by different routes. The aqueous formulations provided herein can be administered using any appropriate method. Administration can be, for example, topical (e.g. transdermal, ophthalmic or intranasal); pulmonary (e.g., by inhalation or insufflation or powders or aerosols); oral, or parenteral (e.g. by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). In some embodiments, the mode of administration is intraperitoneal.

Gram-positive bacteria are bacteria that appear violet or purple after exposure to the Gram stain test. Gram-positive bacteria contain a peptidoglycan layer in the cell wall; the Gram stain binds to or is absorbed by this peptidoglycan layer such that the stain remains after a wash. Examples of Gram-positive bacteria include Actinomyces sp., Bacillus sp., Clavibacter sp., Clostridium sp., Corynebacterium sp., Enterococcus sp., Lactobacillus sp., Leifsonia sp., Listeria sp., Mycoplasma sp., Nocardia sp., Propionibacterium sp., Rathybacter sp., Staphylococcus sp., Streptococcus sp., and Streptomyces sp. Bacillus subtilis is commonly used in experiments as an example for Gram-positive bacteria. Escherichia coli is commonly used in experiments as an example for Gram-negative bacteria.

Mycobacteria are members of the order Actinomycetales, and the only genus in the family Mycobacteriaceae. Currently, the genus Mycobacterium has more than 100 recognized or proposed species, including numerous pathogens and saprophytic organisms of warm-blooded animals. Mycobacteria can be divided into two groups based on growth rate. Rapid growers form visible colonies on solid media often within seven days, while slow growers, take longer to produce visible colonies. Mycobacterium tuberculosis causes tuberculosis in humans, M. bovis causes tuberculosis in cattle and humans, and M. africanum is a rare cause of human tuberculosis in central Africa. While these pathogenic species exhibit some phenotypic differences, they are genetically very similar and are often classed as the ‘Mycobacterium tuberculosis complex’. Other pathogenic mycobacteria include the Mycobacterium avium complex (MAC) and the non-tuberculous mycobacteria (NTM, atypical mycobacteria, or environmental mycobacteria). Both these groups are frequent opportunistic pathogens, particularly in immunosuppressed individuals.

Aqueous formulations of the invention are administered in such amounts and for such time as is necessary to achieve a desired result. As used herein, an “effective” amount of an aqueous formulation is an amount which is suitable to elicit a response effective to treat, attenuate, or prevent disease. Thus, the amount effective to treat, attenuate, or prevent disease, as used herein, refers to a nontoxic but sufficient amount of the aqueous formulation to treat, attenuate, or prevent disease in any subject. For example, the “therapeutically effective amount” can be an amount to treat, attenuate, or prevent infection (e.g., bacterial infection, Mycobacterium infection), etc. The exact amount required to achieve a desired result may vary, depending on the particular components of the formulation, and from subject to subject, depending on the species, age, and general condition of the subject, the stage of the disease, the particular pharmaceutical mixture, its mode of administration, and the like.

The amount of aqueous formulation comprising an antibiotic and DOC in each dose is selected to allow the formulation, when administered as described herein, to induce an appropriate response without significant, adverse side effects. A “protective” response as used herein is a response sufficient to protect a treated subject from productive infection by a particular pathogen or pathogens. Optimal amounts of components for a particular aqueous formulation can be ascertained by standard studies involving observation of appropriate responses in subjects. Following an initial administration of the aqueous formulation, subjects can receive one or several additional doses adequately spaced in time. Such amounts may vary depending upon which bacteria the antibody formulation is administered to treat, and may be formulated in a unit dosage form for ease of administration and uniformity of dosage.

The expression “unit dosage form,” as used herein, refers to a physically discrete unit of aqueous formulation appropriate for the subject to be treated. The specific therapeutically effective dose for any particular subject or organism may depend upon a variety of factors including the severity or degree of risk of infection; the activity of the specific aqueous formulation employed; other characteristics of the specific aqueous formulation employed; the age, body weight, general health, sex of the subject, the diet of the subject, the pharmacokinetic condition of the subject, the time of administration (e.g., with regard to other activities of the subject such as eating, sleeping, receiving other medicines including other vaccine doses, etc.), the route of administration, the rate of excretion of the specific aqueous formulation employed; other antibiotics used in combination or coincidental with the aqueous formulation employed; and like factors well known in the veterinary and medical arts.

For the purposes of the present invention the “subject” may be a vertebrate. In context of the present invention, the term “subject” includes both humans and other animals, particularly mammals, and other organisms. Thus, the herein provided means and methods are applicable to both human therapy and veterinary applications. Accordingly, herein the subject may be an animal such as a mouse, rat, hamster, rabbit, guinea pig, ferret, cat, dog, chicken, sheep, bovine species, horse, camel, or primate. Preferably, the subject is a mammal. More preferably the subject is an animal.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam, sublingually; ocularly; transdermally, or nasally, pulmonary, and to other mucosal surfaces.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include, sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil, glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Aqueous formulation for use in accordance with the present invention may be formulated according to known techniques. An effective amount of an aqueous formulation can be formulated together with one or more pharmaceutically acceptable carrier materials (organic, inorganic, liquid, or solid). In general, pharmaceutically acceptable carriers include solvents, dispersion media, and the like, which are compatible with pharmaceutical administration. For example, materials that can serve as pharmaceutically acceptable carriers include, but are not limited to sugars such as lactose, glucose, dextrose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; polyols such as glycerol, propylene glycol, and liquid polyethylene glycol; esters such as ethyl oleate and ethyl laurate; agar; buffering agents such as magnesium hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as preservatives, and antioxidants can also be present in the composition, according to the judgment of the formulator (see also Remington's Pharmaceutical Sciences, Fifteenth Edition, E. W. Martin (Mack Publishing Co., Easton Pa., 1975).

Aqueous formulation of the present invention can further comprise one or more additional immunomodulatory components such as, e.g., an adjuvant or cytokine, among others. Non-limiting examples of adjuvants that can be used in the vaccine of the present invention include the RIBI adjuvant system (Ribi Inc., Hamilton, Montana, USA), alum, mineral gels such as aluminum hydroxide gel, oil-in-water emulsions, water-in-oil emulsions such as, e.g., Freund's complete and incomplete adjuvants, Block copolymer (CytRx, Atlanta, Georgia, USA), QS-21 (Cambridge Biotech Inc., Cambridge Massachusetts, USA), SAF-M (Chiron, Emeryville California, USA), AMPHIGEN, proprietary oil in water adjuvant (Zoetis, Parsippany, New Jersey, USA), saponin, Quil A (Brenntag Biosector A/S, Ballerup, Denmark), or other saponin fraction, monophosphoryl lipid A, and Avridine lipid-amine adjuvant. Other immunomodulatory agents that can be included in the vaccine of the invention, comprise, e.g., one or more interleukins, interferons, or other known cytokines.

In some embodiments, at least one booster dose of the aqueous formulation taught herein, is administered after an initial administration of the aqueous formulation taught herein. The booster dose may be identical to the aqueous formulation initially used to treat the subject. The booster dose may be administered as early as four hours after initial treatment. In some embodiments, the booster dose may be administered at least one day after initial vaccination.

The response from the initial or booster aqueous formulation taught herein may protect a naive subject from subsequent full-blown infection when exposed to bacterium. Alternatively, administration of the initial or booster dose is used to provide treatment for an existing bacterial infection. The protective response either wholly or partially prevents or arrests the development of symptoms related to bacterial disease or bacterial infection, in comparison to a non-treated control subject, in which disease progression is not prevented.

Aqueous formulations of the present invention can be prepared following accepted convention to include acceptable carriers for animals, including humans (if applicable), such as standard buffers, stabilizers, diluents, preservatives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Other suitable vaccine vehicles and additives, including those that are particularly useful in formulating modified live vaccines, are known or will be apparent to those skilled in the art. See, e.g., Remington's Pharmaceutical Science, 18th ed., 1990, Mack Publishing, which is incorporated herein by reference.

An effective amount of any of the aqueous formulations described herein can be determined by conventional means, starting with a low dose of an aqueous formulation comprising an antibiotic and DOC, and then increasing the dosage while monitoring the effects. An effective amount may be obtained after a single administration of an aqueous formulation comprising an antibiotic and DOC or after multiple administrations of an aqueous formulation comprising an antibiotic and DOC. Known factors can be taken into consideration when determining an optimal dose per animal. These include the presence of other drugs in the animal, the species, size, age, and general condition of the animal, and the like.

The timing of administration of an aqueous formulation comprising an antibiotic and DOC, and the number of boosters, if any, will preferably be determined by a veterinarian or medical doctor based on analysis of all relevant factors, some of which are described above.

Suitable doses for aqueous formulations comprising an antibiotic and DOC according to the practice of the present invention range generally from about 10 mg/kg subject to 500 mg/kg subject per dose, as may be determined by standard methods. When administering to a subject of a very young age, a mucosal delivery route may be preferred.

The effective dose amount of an aqueous formulation comprising an antibiotic and DOC of the present invention can be determined using known techniques, taking into account factors that can be determined by one of ordinary skill in the art such as the weight of the subject to be treated. By way of example, formulations may be delivered orally, parenterally, intradermally, subcutaneously, intramuscularly, intranasally or intravenously. Oral delivery may encompass, for example, adding the compositions to the feed or drink of the animals.

The present invention further provides methods for preparing an aqueous formulation comprising at least one antibiotic and DOC, and optionally at least one carrier and/or excipient.

Additional features and variations of the invention will be apparent to those skilled in the art from the entirety of this application, including the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention. It will be clear that the invention may be practiced otherwise than as particularly described in the foregoing description and examples. Numerous modifications and variations of the present invention are possible in light of the above teachings and, therefore, are within the scope of the invention.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

The invention illustratively disclosed herein suitably may be practiced in the absence of any element (e.g., method (or process) steps or composition components) which is not specifically disclosed herein. Thus, the specification includes disclosure by silence (“Negative Limitations In Patent Claims,” AIPLA Quarterly Journal, Tom Brody, 41(1): 46-47 (2013): “ . . . Written support for a negative limitation may also be argued through the absence of the excluded element in the specification, known as disclosure by silence . . . . Silence in the specification may be used to establish written description support for a negative limitation. As an example, in Ex parte Lin [No. 2009-0486, at 2, 6 (B.P.A.I. May 7, 2009)] the negative limitation was added by amendment . . . . In other words, the inventor argued an example that passively complied with the requirements of the negative limitation . . . was sufficient to provide support . . . . This case shows that written description support for a negative limitation can be found by one or more disclosures of an embodiment that obeys what is required by the negative limitation . . . .”

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this invention, the same will be better understood by reference to certain specific examples, which are included herein only to further illustrate the invention and are not intended to limit the scope of the invention as defined by the claims.

### Example 1

### Preparation of Antibiotic Solutions

Tunicamycins and tunicamycin derivatives were solubilized in water using deoxycholate.

The TunR2 and TunR1 used were purchased from Cayman Chemical Company (Ann Arbor, Michigan, USA), or were prepared from tunicamycin by catalytic hydrogenation as described in Price NPJ et al. (2017, “Modified tunicamycins with reduced eukaryotic toxicity that enhance the antibacterial activity of β-lactams,” J. Antibiot. 70(11) 1070-1077). Briefly, TUN (10 mg, Sigma-Aldrich) was dissolved in methanol:toluene (1:1 v/v, 2 mL), plus glacial acetic acid (10 μl). A catalytic amount of solid palladium on carbon catalyst (10% Pd/C, 3 mg) was added followed by an addition of solid sodium borohydride (7 mg). The reaction was capped and held at 25° C. for 30 minutes. This was then filtered through a pad of Celite filter aid to remove the spent Pd/C catalyst. The filtrate was evaporated to dryness, and re-dissolved and refluxed in methanol (2 mL, 80° C., 15 minutes). This latter step is to convert the residual sodium borohydride to methyl borate and residual acetic acid to methyl acetate, which were subsequently removed by evaporation. The material produced upon evaporation is the single reduced TunR1. Control reactions were run that either lacked the Pd/C catalyst (but included the borohydride) or lacked both Pd/C and the borohydride. The reaction was then optimized for co-reduction of the uridyl 5,6-double bond, initially using uridine as a model compound. Using the condition above at higher temperature and a longer reaction time (60° C., 50 hours) resulted in complete reduction of the uridine double bond. This optimized reaction was then applied to commercial TUN to effect the double reduction of the 2′″,3′″-alkene and 5,6-uridyl double bonds. Cooling and work up as described above gave the double-reduced TunR2.

Sodium deoxycholate (DOC, 5β-cholan-24-oic acid-3α, 12α-diol sodium salt) was purchased from Sigma-Aldrich, St. Louis, Missouri, USA. All other reagents were of analytical grade and used without further purification.

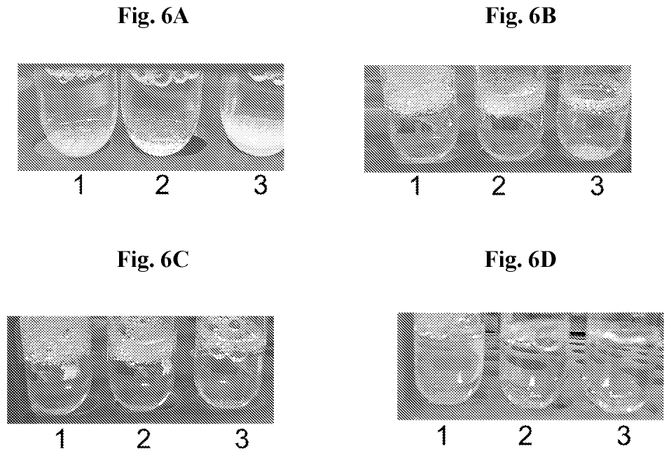

The modified tunicamycin TunR2 solid powder (8 mg, 8 mmoles) and DOC solid powder (4.4 mg, 10 mmoles) were vortexed together in an equimolar ratio, and was prepared in water for injection (3 mL) and stirred until dissolved. If needed, the pH was adjusted to 5.8 by addition of 10% acetic acid. The TunR2 was initially insoluble under these conditions, but began to dissolve after 5 minutes at 50° C. (in the range of about 50° C. to about 70° C.) and was completely soluble after 10 minutes at this temperature (FIG. 6A to FIG. 6C). The TunR2-DOC preparations were water-soluble and gave clear solutions in water similar in appearance to the DOC standard. Under the same conditions, natural, unmodified tunicamycin was faster to dissolve and gave a clear solution within 5 minutes at 50° C., and a second chemically modified tunicamycin (TunR1) dissolved immediately in the 5% aqueous DOC without requiring heating (FIG. 6A to FIG. 6C). Hence, all three tunicamycin-based compounds were completely solubilized in water when mediated by emulsification with DOC. The TunR1 and TunR2 solutions were also stable after cooling, and remained in solution even after standing for 18 hours at room temperature (FIG. 6D). The native tunicamycin solution became somewhat hazy after this time, but was readily clarified on gentle re-warming.

This example shows that tunicamycin, TunR1, and TunR2 can be solubilized in water using deoxycholate (DOC) in the temperature range of about 50° C. to about 70° C. At low temperatures the tunicamycin, TunR1, and TunR2 are insoluble in the DOC/water. The aqueous solutions prepared in this way are stable at room temperature for at least one month.

### Example 2

### Solution Characterization Methods

It was important to show that dissolving the tunicamycins, TUNR1, or TunR2 in deoxycholate (DOC) did not alter their chemical structures in any way. A chemical alteration might improve solubility in water, for example, but if so the hypothetical alteration might also change the structure and biological activity of the tunicamycins, TUNR1, or TunR2.

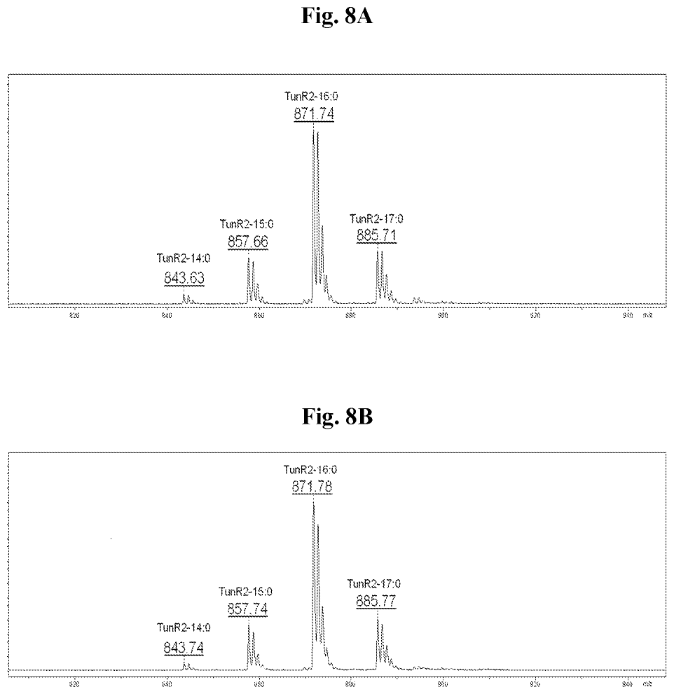

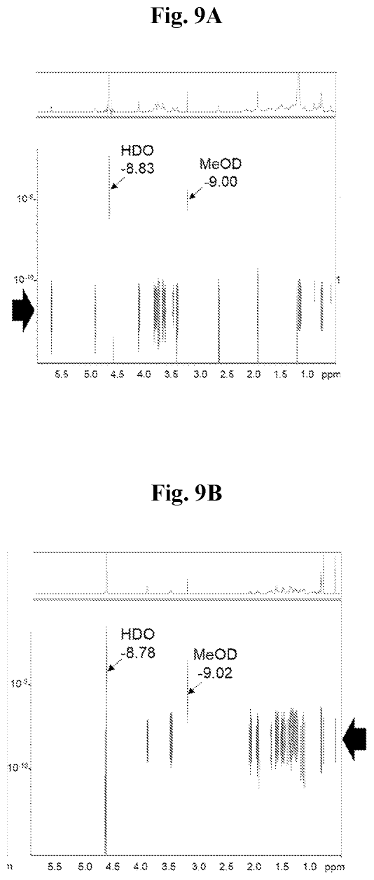

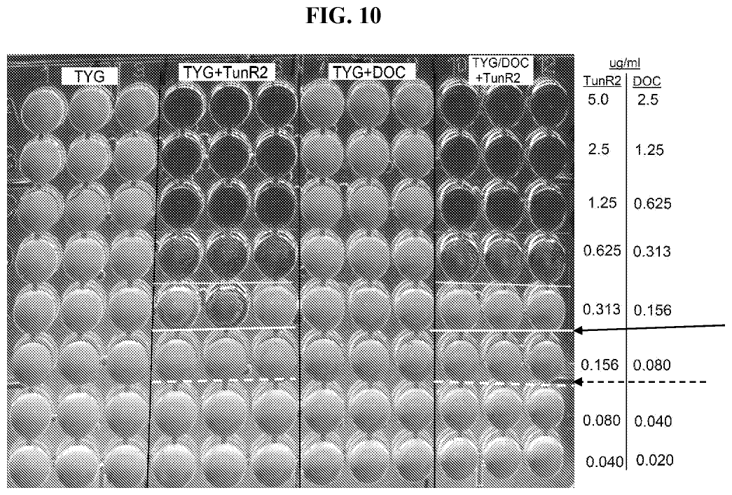

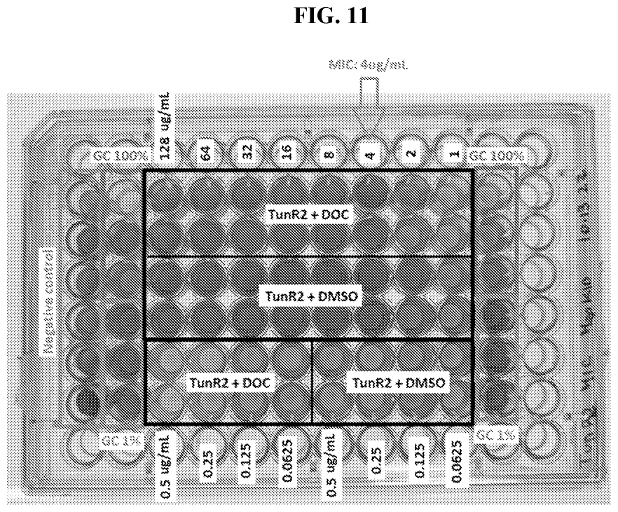

This example shows that the tunicamycins, TUNR1, and TunR2 are not chemically altered in any way by the DOC treatment. Matrix-assisted Laser Desorption/Ionization Mass spectrometry (MALDI-TOF-MS), HPLC/MS, and various 1D and 2D nuclear magnetic resonance spectroscopy techniques showed the structures before and after the DOC treatment to be unchanged (see FIG. 8A, FIG. 8B, FIG. 9A, and FIG. 9B). Diffusion orientated spectroscopy (DOSY-NMR) was used to show that the tunicamycins, TUNR1, and TunR2 form a non-covalent complex with the DOC, which allows them to dissolve in water without physically altering their chemical structures (see FIG. 9A and FIG. 9B). In addition, the biological activities (minimal inhibitory concentrations (MIC) and minimal biofilm inhibitory concentrations (MBIC)) of the tunicamycins, TunR1, or TunR2 were also unaltered by the DOC treatment as shown in FIG. 10 and FIG. 11.

### 1D) and 2D-NMR Spectroscopy—

1H and 2D NMR (COSY, HSQC, and HMBC) spectra were recorded on a Bruker Avance III 500 MHz spectrometer operating at 125 MHz. The spectra were obtained in methanol-d4 or DMSO-d6 using residual solvent signals as internal standards. Diffusion orientated (DOSY) NMR spectra were obtained on a JEOL 600 mHz instrument using DOSY pulse sequences provide by the manufacturer.

### Matrix-Assisted Laser Desorption Ionization Mass Spectrometry—

The TUN components were verified by molecular mass using MALDI-TOF mass spectrometric analysis on a Bruker-Daltonics Microflex instrument (Bruker-Daltonics, Billerica, Massachusetts, USA) as described previously (K. N. Gangadhar, et al., 2014, “Synthesis and evaluation of sodium deoxycholate sulfate as a lipid drug carrier to enhance the solubility, stability and safety of an amphotericin B inhalation formulation,” Int. J. Pharm. 471 430-438). Briefly, samples were diluted 1:1 v/v with 2,5-dihydroxybenzoate matrix (saturated solution in acetonitrile; typically 1-2 μl) and spotted onto a standard 100-place stainless steel target. The MS spectra were acquired in reflectron mode with positive ion detection, averaging 2000 shots at 60-70% laser power (150 μJ maximum output, 337 nm). A pulsed ion extraction (200 ns) was used with the ion sources set to 19.0 kV and 13.6 kV, respectively.

### HPLC Method—

The HPLC system used was a Finnigan Surveyor (ThermoFisher Scientific, W. Palm Beach, Florida, USA) using a reversed-phase C30 column specifically designed to separate long chain carotenes (YMC Carotene C-30, 4.6×250 mm; 3 μm particle size) (YMC America Inc., Allentown, Pennsylvania, USA). The various TUN N-acyl variants were resolved using an acetonitrile—water gradient (45-100%; 1 mL·min⁻¹; 27° C.) and typically eluted in order of increasing acyl chain length between 5-11 min. The peaks were monitored by PDA detector and were collected manually from multiple injection runs.

Antimicrobial activities—minimal inhibitory concentrations (MIC) and minimal biofilm inhibitory concentrations (MBIC)—the biological activities (MICs and MIBCs) of the TunR2 compounds were tested on various microorganism (FIG. 11). These bioactivities were compared with the compounds dissolved in either DMSO or in DOC/water, and the MICs and MIBCs were shown to be identical. In addition, comparative biological activities of the compounds dissolved in either DMSO or DOC/water were compared against the cattle Johne's disease pathogen Mycobacterium avium subsp. tuberculosis (MAP) (FIG. 12)

Seed cultures of Bacillus subtilis ATCC 6633, Escherichia coli ATCC 9637, Mycobacterium smegmatis mc² 155, and Saccharomyces cerevisiae NRRL Y-139 were started from freezer stocks and grown in tryptone-yeast extract-glucose (TYG) broth for 24 hours at 27° C. Microbial dilutions were prepared as follows. Sterile PBS (phosphate buffer saline, 1 mL) was added to a glass tube using a disposable 10 mL pipet. Using a sterile loop microbial material was transferred from an agar plate, and the turbidity was measured and adjusted to 1.0 Macfarlane units. The inoculum dilution was then prepared. To a sterile 50 mL blue-capped centrifuge tube, 50 mL TYG stock media were added using a 25 mL disposable pipet. To this tube was then added 1 mL of the microbial dilution, and the contents were mixed by vortexing (this will give a 1:50 v/v dilution). This 1:50 v/v dilution inoculum was dispensed using a multichannel pipet, adding 100 μL to each well of a 96-well round-bottom microtiter plate.

MICs and MBICs were evaluated using two-fold broth dilution series in 96-well round-bottom microtiter plates. Diluted solutions were prepared by adding 20 μl of the 1 mg/mL stock of the TunR2 to 1.980 mL of TYG in a 15 mL blue-capped centrifuge tube for a total of 2 mL volume at 10 μg/mL. This was repeated for the other test compounds (tunicamycin, TunR1, sodium deoxycholate, and TunR2-DOC). A blank of 20 μl dimethyl sulfoxide (DMSO) up to 2 mL TYG was also included. The microtiter plates were then set up as follows. To wells 1, 2, and 3 of the first row (A row) of the microtiter plate 200 μl of TYG media control were added. To wells 4, 5, and 6 in row A of the plate 200 μl TYG+TunR2 (10 μg/mL) were added. To wells 7, 8, and 9 in row A of the plate, 200 μl TYG+DOC (5 μg/mL) were added. And to wells 10, 11, and 12 in row A of the plate, 200 μl TYG+TunR2 (10 μg/mL)+DOC (5 μg/mL) were added. To the remaining wells of the microtiter plate 100 μl of the sterile TYG liquid media were added.

Two-fold dilutions were then prepared as follows. One hundred (100) μl of the row A contents that contain the test compound were transferred to the next row and mixed 5 times. Then 100 μl of this were taken and transferred to the next row, and mixed 5 times. This transferring and mixing was repeated to row G, leaving row H as a control row. This is a serial dilution where row A=5 μg/mL final concentration, row B=2.5 μg/mL, row C=1.25 μg/mL, row D=0.625 μg/mL, row E=0.313 μg/mL, row F=0.156 μg/mL, row G=0.080 μg/mL, and row H=0.040 μg/mL. Each well will contain 200 μl total volume at this stage. A lid was then put on the microtiter plates, and they were placed in a PERSPEX thermoplastic acrylic resin box so that they would not dry out. The box was placed into the 37° C. incubator and microbial growth and biofilm formation were assessed visually on a daily basis for six days.

Comparative Minimal Inhibitory Concentrations (MIC) on the Johne's Disease Pathogen Mycobacterium avium Subsp. Tuberculosis (MAP)—

Tunicamycin stock solutions (10 mg/mL): A. Deoxycholate (DOC): 10 mg Tun, TunR1, or TunR2+5 mg sodium deoxycholate (DOC), then 1 mL dH₂O were added. The mixture was warmed at 60° C. for 10 minutes, until soluble. Mixture allowed to cool down to RT. Mixture sterilized by transferring through a 0.2 μm filter. Material used within 2 hours or aliquoted into 0.1-0.2 mL/vial and stored at −20° C. B. DMSO: 10 mg Tun, TunR1, or TunR2+1 mL DMSO. Mixture sterilized by transferring through a 0.2 μm filter. Used immediately, or aliquoted into 0.1-0.2 mL/vial and stored at −20° C. to 4° C. For each agent, MIC determinations were done by testing at least 8 concentrations in separate wells to cover the full range of potential MIC values (128, 64, 32, 16, 8, 4, 2, 1, 0.5, 0.25, 0.125, 0.0625 μg/mL) in triplicate.

Inoculum was prepared from growing broth pellets by centrifugation at 4000 rpm for 20 minutes (1550 g force) and resuspension in 5 mL PBS, using a 25G needle to disrupt the clumps for 2 minutes, waiting 30 minutes for the remaining clumps to settle. The turbidity of the supernatant was adjusted to OD₆₀₀=0.2-0.25 (1 McFarland) by addition of sterile PBS. After vortexing for 30 seconds, a 1:25 dilution of the bacterial suspension was prepared in 7H9/OADC broth by two steps of tenfold dilutions. The volume of bacterial suspension required for one test plate is 10 mL. A 10-1 suspension was prepared by adding 0.4 mL of the 1 McFarland bacterial suspension to 9.6 mL of 7H9/OADC and vortexing until swirling for at least 30 seconds (OD₆₀₀=0.01) was obtained. This is the final inoculum or growth control.

The MICs were determined by a modified European Committee on Antimicrobial Susceptibility Testing (EUCAST) procedure. Isolates of the MAP were grown on solid media or broth and sampled from fresh cultures. Strains were grown on Broth 7H9/OADC: Middlebrook 7H9 medium (7H9)+2 mg/mL mycobactin J+10% OADC without TWEEN 80 surfactant in U-bottom microtiter plates. One hundred (100) μl 7H9/OADC were added to all wells, except the peripheral wells (rows A and H, column 12), which were filled with sterile distilled water to prevent desiccation during the incubation time. One hundred (100) μl of the 4× working solution were added to the wells corresponding to the highest concentration of each agent. Using a multichannel pipette 1:2 dilutions were made by adding 100 μl of the antibiotic solution present in the highest concentration row to the following row and finally discarding the last 100 μl of the last row/wells. Also, the highest concentration of the drug was added into three negative controls wells. After inoculation, the plates were covered with an adhesive sterile film for culture plates (porous, VWR, #60941-086), then covered with the plastic lid, and placed in O₂-/CO₂-permeable plastic bags in a humidify chamber). Plates were incubated at 38° C.±1° C.

### Visual Growing MIC Determination:

The U-bottom plates were read using an inverted mirror, first after 10 to 12 days of incubation and then at 21 days of incubation, or until the visual growth of the GC1%. The negative control should showed no growth for the test to be valid. If the GC100% was positive, the GC1% was checked. If the GC1% also showed visible growth (usually weaker positivity than GC100%), MIC was determined as the lowest concentration of the agent where no visible growth was observed. If there was insufficient growth of the GC1% after 21 days, plates were incubated until a maximum of 30 days. Also, MIC was determined by OD₆₀₀ measurement in flat-bottom plates (without adhesive film), if the strains were not clumpy, MIC₉₀ and MIC₅₀, or dose-response curve were calculated using the following formula.

$$\% inhibition = 1 - [ ( {OD}_{sample} - {OD}_{neg} ) / ( {OD}_{GC 100 \%} - {OD}_{neg} ) ] * 100$$

### REMA MIC Determination:

Resazurin is a redox indicator that changes color (from blue/purpure to pink) when bacteria consume O₂ and therefore allows growth quantification. The flat-bottom plates were checked using an inverted mirror, first after 7 to 10 days of incubation, 30 μl of resazurin were added in one of the GC100% and GC1% wells and the plates incubated one day more. If color change was evident, 30 μl of resazurin were added to the rest of the wells and the plates re-incubated for 24 hours more for the final reading. Resazurin was not included in the wells before the incubation to avoid inhibition of mycobacterial growth. If color change was not evident, the plate was incubated for another 5 to 7 days, incubated until a maximum of 14 to 21 days. The MIC is defined as the lowest concentration of the drug that prevented this change in color. A change in color of growth control well to pink indicated the proper growth of the isolate and no change in color of sterile control well indicated absence of contaminants. To determinate MIC (MIC90 and MIC50, or dose-response curve) by fluorescence index 96-well black flat-bottom FLUORONUNC black polystyrene plates designed for fluorescent assays were used (without adhesive film) (Resazurin: Ex.535 nm; Em.590 nm). For each strain, a positive control and a negative control was used. The positive control was cells grown without antibiotic, to determine the maximum fluorescence that could be obtained. The negative control was medium plus antibiotic without cells. The percent (%) inhibition was calculated using the formula below.

$$\% inhibition = 1 - [ ( {F}_{sample} - {F}_{neg} ) / ( {F}_{GC 100 \%} - {F}_{neg} ) ] * 100$$

The results show that TunR2-type antibiotics are solubilized in water by complexation with deoxycholate by gentle warming (about 50° C. to about 70° C.) to form TunR2-deoxycholate (TunR2-DOC) micelles. Chemical analysis showed that the treatment of the DOC does not alter the chemical structure, and DOSY spectroscopy show that the (TunR2-DOC) micelles are a water-soluble, non-covalent complex. Bioactivity testing on various microbes including a pathogenic Johne's disease Mycobacterium strain, show that the TunR2-DOC micelles dissolved in water have the same activity as the same concentration of TunR2 dissolved in DMSO solvent. Hence, TunR2-DOC micelles in water are an aqueous-based carrier system that can be used as a direct replacement for the toxic DMSO or MeOH carriers.

### Example 3

### Characterization

The following example shows that dissolving the tunicamycins, TunR1, or TunR2 with deoxycholate (DOC) in water did not alter their chemical structures in any way. The tunicamycins, TunR1, and TunR2 formed non-covalent micelles with deoxycholate (DOC), and it is these micelles that are soluble in water or aqueous buffers.

### Physical Properties of TunR2-Deoxycholate Micelles—

The present work undertaken to develop a water-soluble TunR2 formulation solubilized by sodium deoxycholate to reduce the solvent toxicity and to improve the physiochemical properties. Solutions were prepared either by mixing together TunR2 and DOC powders in equimolar portions and adding water, or by preparing a DOC aqueous stock solution and adding this directly to the TunR2. At 10 mg/mL, the TunR2 was initially insoluble under these conditions, but began to dissolve after 5 minutes at 50° C. and was completely soluble after 10 minutes at this temperature. The TunR2-DOC preparations were water-soluble and gave clear solutions in water similar in appearance to the DOC standard. Under the same conditions, natural, unmodified tunicamycin was faster to dissolve and gave a clear solution within 5 minutes at 50° C., and a second chemically-modified tunicamycin (TunR1) dissolved immediately in the 5% aqueous DOC without requiring heating. Hence, all three tunicamycin-based compounds tested are completely soluble in water when mediated by emulsification with DOC. The TunR1 and TunR2 solutions were also stable after cooling, and remained in solution even after standing for 18 hours at room temperature. The native tunicamycin solution became somewhat hazy after this time, but was readily clarify on gentle re-warming.

As seen in FIG. 6A, crystals of native tunicamycin (1); TunR1(2); and TunR2(3) were visible after the addition of water and sodium deoxycholate. As seen in FIG. 6B, after heating for 5 minutes at 50° C., no crystals are detected in the native tunicamycin (1) or TunR1(2), but some crystals were still present in the TunR2 (3) sample. As seen in FIG. 6C, after heating for 10 minutes at 50° ° C. all three antibiotics were completely dissolved. As seen in FIG. 6D, all three antibiotics remained in solution after cooling at room temperature for 18 hours.

Because of the steroid-like structure of deoxycholate it was expected that foaming would be minimal, and indeed this was the case for the TunR1 and TunR2 preparations shown in FIG. 6A to FIG. 6D. Some foaming was apparent for the native tunicamycin solution, but this was relatively minor and is not expected to be problematic for subsequent handling or usage.

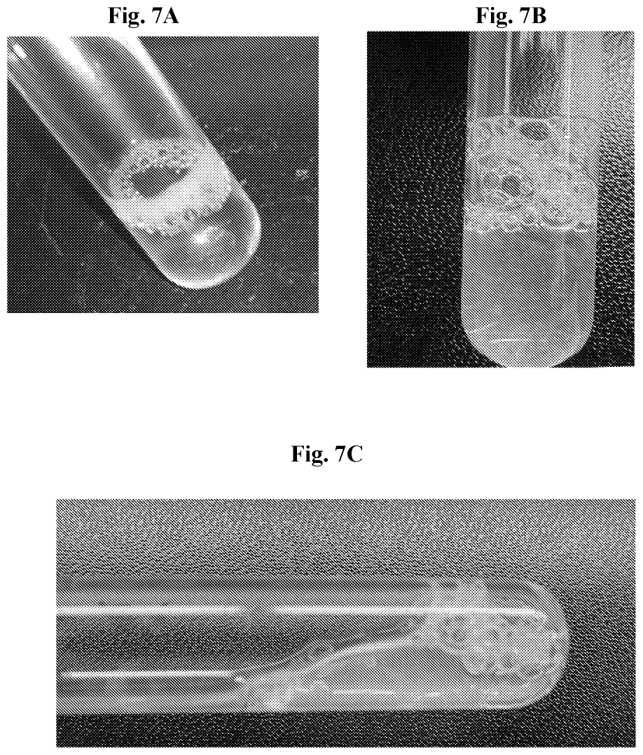

Gel formation with DOC has been observed previously. Although DOSY-NMR experiments also showed that the gelation has little effect on the aqueous diffusion of free DOC (Li P et al, 2022, “Using nuclear magnetic resonance spectroscopy to probe hydrogels formed by sodium deoxycholate,” Langmuir 38: 5111-5118). FIG. 7A to FIG. 7C depict images of the complexation gelling of TunR2-DOC micellar structure. FIG. 7A shows a picture of an equimolar solution of TunR2 and Doc (10 mM) in 1 mL deionized water. FIG. 7B shows a picture of gelling after 24 hour freeze-thawing with the tube held vertically. FIG. 7C shows a picture of gelling after 24 hour freeze-thawing with the tube held horizontally.

The data showed that the TunR2-DOC complex in stable in solution for at least 42 days. MALDI-TOF/MS spectra of TunR2 dissolved in aqueous solution with deoxycholate is shown in FIG. 8A and FIG. 8B. An equimolar solution of TunR2+DOC (10 mM) in 1 mL deionized water was prepared 1:1 v/v with MALDI matrix (2,5-dihydroxybenzoic acid) in acetonitrile. The expected TunR2 components are TunR2-14:0 (m/z 843.63), TunR2-15:0 (m/z 857.66), TunR2-16:0 (m/z 871.74) and TunR2-17:0 (m/z 885.71), are identical to those obtained from TunR2 in DMSO solution (Price et al, 2017, Supra). FIG. 8A shows an MS spectrum acquired 9/30/22;

FIG. 8B shows an MS spectrum acquired 11/3/22, after 42 days of room temperature.

Diffusion-Orientated Spectra (DOSY-NMR) of the TunR2-DOC complex (log D=−10.28 m/s2) and the control deoxycholate (log D=−9.66 m/s2) in D20 (10 mM) are shown in FIG. 9A and FIG. 9B. The diffusion constants (log D) of residual deuterated water (HDO) and methanol (MeOD) are also indicated on the graphs. The corresponding ¹H-NMR spectra are shown above the graphs. All spectra were acquired at 600 MHz.

This example shows that tunicamycins, or TUNR1, or TunR2 form non-covalent micelles with deoxycholate (DOC), and it is these micelles that are soluble in water or aqueous buffers. The TunR2-DOC micelles were visualized by diffusion orientated NMR and shown to have a measured diffusion constant of log D=−10.28 m/s2.

### Example 5

### Antibacterial Activity

The MIC₅₀ and MIC₉₀ values of antibiotic/DOC micelles were tested.

With the increasing incidence of multi-drug resistant bacterial infections, it is essential to redesign established antimicrobial agents for clinical and veterinary use. Tested as a control in the MIC assays was DOC at the same concentration as the TunR2. The data showed that DOC at these concentrations had no effect against Mycobacterium smegmatis. Previously, the MICs of the TunR2 standard were investigated and showed that the MICs for Mycobacterium smegmatis were in the range of 0.5 to 2 μg/mL and the MBIC 2-fold higher than this (J. Hering et al., 2020, “Exploring the active site of the Antibacterial Target MraY by Modified Tunicamycins,” ACS Chem. Biol. 15: 2885-2895).

The minimal inhibitory concentration (MIC) data and the minimal biofilm inhibitory concentration data obtained are summarized in Table 1 below.

TABLE 1
Minimal Inhibitory Concentrations and Minimal
Biofilm Inhibitory Concentrations

| | | TunR2 | TunR2- | Aqueous |
|---|---|---|---|---|
| | TYG | in | Doc in | DOC |
| | Control | DMSO | water | control |
| Bacillus subtilis | — | 0.3 | 0.3 | >2.5 |
| Escherichia coli | — | >5.0 | >5.0 | >2.5 |
| Mycobacterium smegmatis | — | 0.3/0.6 | 0.3/0.6 | >2.5 |
| Saccharomyces cerevisiae | — | >5.0 | >5.0 | >2.5 |

Minimal inhibitory (MIC) data for the TunR2-DOC complex against the model TB organism Mycobacterium smegmatis is shown in FIG. 11. This figure shows a picture of a 1:1 equimolar dilution series of TunR2-DOC (5.0-0.04 μg/mL of TunR2) in TYG medium in triplicate wells in a microtiter plate. Growth and biofilm formation (opaque wells) are shown after 6 days at 32° C. The minimal inhibitory concentration (MIC, 1.25 μg/mL) is as denoted by the solid white lines and solid arrow, and the minimal biofilm inhibitory concentration (MBIC, 0.625 μg/mL) is indicated by white dash lines and dashed arrow. As seen in the figure, MIC and MBIC are unaffected by the inclusion of the DOC. Control lanes are TYG medium only, and TYG+DOC gradient (2.5-0.02 μg/mL).

The results show high potency of TunR2-DOC against M. smegmatis (F12 contains ˜57% TunR2), comparable to TunR2 dissolved in DMSO but not to the DOC molecules. This shows that the DOC molecule did not affect the activity of the TunR2 molecules, and that the antimicrobial activity of the TunR2 against Mycobacterium smegmatis (mycobacterium), Bacillus subtilis (Gram positive bacterium), and Escherichia coli (Gram negative bacterium), and Saccharomyces cerevisiae (yeast, model eukaryote) are identical with the TunR2 activities measured in DMSO solution. Data is shown in FIG. 12, and above in Table 1.

We claim:

1. An aqueous formulation comprising at least one antibiotic and sodium deoxycholate (DOC), and optionally comprising at least one carrier and/or excipient.

2. The aqueous formulation of claim 1, wherein the at least one antibiotic has poor water solubility.

3. The aqueous formulation of claim 2, wherein the at least one antibiotic is a tunicamycin, a modified tunicamycin, capazamycin, liposidomycin, muraminomicin, polymyxin B, nikkomycin Z, a lipophilic antifungal polyene antibiotic, or a mixture thereof.

4. The aqueous formulation of claim 3, wherein at least one antibiotic is a modified tunicamycin.

5. The aqueous formulation of claim 4, wherein the modified tunicamycin is TunR2.

6. The aqueous formulation of claim 4, further comprising a β-lactam antibiotic, a non-β-lactam antibiotic, or a combination thereof.

7. The aqueous formulation of claim 6, wherein the β-lactam antibiotic is a penicillin, a cephalosporin, a monobactam, a carbapenem, or a combination thereof.

8. A method for treating a subject in need thereof, the method comprising administering to the subject an effective amount of an aqueous formulation of claim 1.

9. A method for treating a subject in need thereof, the method comprising administering to the subject an effective amount of an aqueous formulation of claim 6.

10. A stable antibiotic/DOC complex.

11. The stable antibiotic/DOC complex of claim 10, wherein the antibiotic is a tunicamycin, a modified tunicamycin, capazamycin, liposidomycin, muraminomicin, polymyxin B, nikkomycin Z, amphotericin B, candicidin, filipin, hamycin, natamycin, nystatin, rimocidin, or a mixture thereof.

12. The stable antibiotic/DOC complex of claim 11, wherein the antibiotic is a modified tunicamycin.

13. A method for treating or preventing a bacterial infection in an animal, the method comprising administering to the animal an effective amount of an aqueous composition comprising at least one antibiotic and sodium deoxycholate (DOC), and optionally comprising at least one carrier and/or excipient.

14. The method of claim 13, wherein the at least one antibiotic is a tunicamycin, a modified tunicamycin, capazamycin, liposidomycin, muraminomicin, polymyxin B, nikkomycin Z, amphotericin B, candicidin, filipin, hamycin, natamycin, nystatin, rimocidin, or a mixture thereof.

15. The method of claim 13, wherein the bacterial infection is caused by Gram positive bacteria.

16. The method of claim 13, wherein the bacterial infection is caused by mycobacterium.

17. A method for preparing an aqueous antibiotic-DOC composition, the method comprising mixing equimolar ratios of a solid antibiotic with solid DOC in water, and heating while mixing for a sufficient amount of time for the solids to dissolve, and optionally adjusting pH of the aqueous antibiotic-DOC composition.

18. The method of claim 15, wherein the antibiotic is a tunicamycin, a modified tunicamycin, capazamycin, liposidomycin, muraminomycin, polymyxin B, nikkomycin Z, amphotericin B, candicidin, filipin, hamycin, natamycin, nystatin, rimocidin, or a mixture thereof.
